(19) United States
(12) Patent Application Publication
(10) Pub. No.: US 2024/0099997 A1
(43) Pub. Date: Mar. 28, 2024

(54) METALLOENZYME MODULATORS

(71) Applicant: Regents of the University of Minnesota, Minneapolis, MN (US)

(72) Inventors: Ambika Bhagi-Damodaran, Minneapolis, MN (US); Leonardo Rander Asse, Jr., Minneapolis, MN (US); Sourav Chatterjee, Minneapolis, MN (US)

(21) Appl. No.: 18/237,360

(22) Filed: Aug. 23, 2023

Related U.S. Application Data
(60) Provisional application No. 63/400,672, filed on Aug. 24, 2022.

(51) Int. Cl. A61K 31/15; A01N 33/26; A01N 43/40; A01N 43/78; A01N 43/84; A01N 43/90; A61K 31/428; A61K 31/4365; A61K 31/439; A61K 31/445; A61K 31/5375; A61K 31/5377; A61K 31/54; A61P 31/06

(52) U.S. Cl. CPC A61K 31/15; A01N 33/26; A01N 43/40; A01N 43/78; A01N 43/84; A01N 43/90; A61K 31/428; A61K 31/4365; A61K 31/439; A61K 31/445; A61K 31/5375; A61K 31/5377; A61K 31/54; A61P 31/06

(57) ABSTRACT

Disclosed herein are methods to inhibit a metalloprotein and methods to treat diseases and crop infections with a compound of formula I:

X-L-Y I

or a salt thereof. Also disclosed are novel compounds of formula I or a salt thereof and compositions comprising a compound of formula I.

### PRIORITY OF INVENTION

This application claims priority from U.S. Provisional Patent Application No. 63/400,672, filed Aug. 24, 2022. The entire content of this United States Provisional Patent Application is hereby incorporated by reference herein.

### BACKGROUND OF THE INVENTION

More than thirty percent of all proteins utilize metal cofactors for their structural stability and function, but less than four percent of FDA-approved drugs target metalloproteins. Despite significant therapeutic potential as indicated by the success of metalloprotein targeting drugs such as fluconazole, the chemical space of metalloprotein inhibitors has been largely unexplored. In particular, the development of compounds that target the metal centre of therapeutically relevant enzymes often runs into challenges of off-target interactions with other metalloproteins.

Metalloproteins are at the heart of numerous biological processes ranging from cellular signalling and gene regulation to natural product biosynthesis. They maintain mammalian redox homeostasis, disruption of which can cause cancer and neurodegenerative diseases (Gialeli, C., et al., FEBS J. 278, 16-27, 2011; Brown, D. R., Metallomics 2, 186-194, 2010). They also play key roles in redox signalling pathways of various fungal, bacterial, and viral infections (Chen, P. R., et al., Antioxid. Redox Signal. 14, 1107-1118, 2011). For these reasons, there has been longstanding interest in exploring metalloproteins as potential therapeutic targets (Chen, A. Y., et al., Chem. Rev. 119, 1323-1455, 2019; Rouffet, M., et al., Dalton Trans. 40, 3445-3454, 2011). A potent strategy to inhibit metalloproteins involves targeting the active site metal. This approach has led to several FDA-approved metal binding drugs for zinc-based metalloproteins like histone deacetylase (HDAC) and angiotensin-converting enzyme (ACE) (Rouffet, M., et al., Dalton Trans. 40, 3445-3454, 2011; Cohen, S. M., Ace. Chem. Res. 50, 2007-2016, 2017). Such metal binding inhibitors for heme-based metalloproteins would be transformative as numerous heme proteins including cyclooxygenases (Rouzer, C. A., et al., J. Lipid Res. 50, S29-S34, 2009), soluble guanylate cyclase (Horst, B. G., et al., Nitric Oxide Biol. Chem. 77, 65-74, 2018), and fungal/bacterial cytochrome P450 (Yoshida, Y., Curr. Top. Med. Mycol. 2, 388-418, 1988); Ouellet, H., et al., Arch. Biochem. Biophys. 493, 82-95, 2010) are high-value therapeutic targets. However, a major hurdle towards the development of metal binding inhibitors for heme is the promiscuity of hemoglobin (Hb) and myoglobin (Mb) at millimolar concentrations (Otto, J. M., et al., Haematologica 102, 1477-1485, 2017) that makes selective targeting difficult to achieve.

Accordingly, there is a need for new methods and/or therapeutics to treat diseases including new methods and/or therapeutics that target (e.g., inhibit) metalloproteins (e.g., heme enzymes). There is also a need for new methods and/or compounds that are useful as pesticides including new methods and/or compounds that are useful as pesticides that target (e.g., inhibit) metalloproteins (e.g., heme enzymes).

### SUMMARY OF THE INVENTION

Methods and compounds disclosed herein modulate (e.g., inhibit) metalloproteins (e.g., metalloenzymes such as heme enzymes) and thus may be useful to treat diseases and or conditions (e.g., diseases and or conditions in a mammal such as a human), wherein a metalloprotein is implicated.

Accordingly, one embodiment provides a method to inhibit a metalloprotein (e.g., in vitro and/or in vivo) comprising contacting the metalloprotein with a compound of formula I:

X-L-Y I

or a salt thereof, wherein:
X is nitrite, hydrazine, —SCN, —NCS, or —OCN;
L is a linker or L is absent; and
Y is a moiety that binds to a gas tunnel or a substrate tunnel of the metalloprotein.

One embodiment provides a method to inhibit a metalloprotein (e.g., in vitro and/or vivo) comprising contacting the metalloprotein with a compound comprising a nitrile or hydrazine and a gas tunnel binding moiety or substrate tunnel binding moiety.

One embodiment provides a method to inhibit a metalloprotein (e.g., in vitro and/or in vivo) comprising contacting the metalloprotein with a compound comprising a nitrite or hydrazine and a gas tunnel binding moiety.

One embodiment provides a method to treat a bacterial disease, viral disease, cardiovascular disease, neurological disease, or cancer in a mammal (e.g., a human) in need thereof, comprising administering a therapeutically effective amount of a compound of formula I or a pharmaceutically acceptable salt thereof as described as herein to the mammal in need thereof.

One embodiment provides a pharmaceutical composition comprising a compound of formula I or a pharmaceutically acceptable salt thereof as described herein, and a pharmaceutically acceptable vehicle.

One embodiment provides a compound of formula I or a pharmaceutically acceptable salt thereof as described as herein, for inhibiting a metalloprotein.

One embodiment provides a compound of formula I or a pharmaceutically acceptable salt thereof as described as herein, for the prophylactic or therapeutic treatment of a disease such as a bacterial disease, viral disease, cardiovascular disease, neurological disease, or cancer.

In one embodiment the disease is caused by pathogens like M. tuberculosis, Klebsiella pneumoniae, Candidiasis, Aspergillosis, bovine rhinotracheitis virus, and bovine viral diarrhea virus.

One embodiment provides the use of a compound of formula I or a pharmaceutically acceptable salt thereof as described as herein, for the preparation of a medicament for inhibiting a metalloprotein.

One embodiment provides the use of a compound of formula I or a pharmaceutically acceptable salt thereof as described as herein, for the preparation of a medicament for treating a bacterial disease, viral disease, cardiovascular disease, neurological disease, or cancer.

One embodiment provides a novel compound of formula I or a salt thereof.

One embodiment provides a compound of formula I or a salt thereof as described herein, provided the compound is not a compound as shown in FIG. 2B or FIG. 4.

One embodiment provides a method to treat a crop to prevent infection of the crop, comprising contacting the crop with a compound of formula I or a salt thereof, wherein the infection is a bacteria infection, an insect infection, a pest infection or another pathogen infection.

One embodiment provides a method to treat infection of a crop, comprising contacting the crop with a compound of formula I or a salt thereof, wherein the infection is a bacteria infection, an insect infection, a pest infection or another pathogen infection.

In one embodiment the crop is maize, potato, rice, soybean, or wheat. In one embodiment the infection is virus like (Barley yellow dwarf luteovirus, Citrus tristeza closterovirus, Plum pox potyvirus). In one embodiment the infection is bacteria like (Erwinia, Pectobacterium, Pantoea, Agrobacterium, Pseudomonas, Ralstonia, Burkholderia, Acidovorax, Xanthomonas, Clavibacter, Streptomyces, Xylella, Spiroplasma, and Phytoplasma). In one embodiment the infection is fungi like (Albugo candida, Plastnodiophora brassicae, Pythium species and mildews).

### BRIEF DESCRIPTION OF THE FIGURES

The patent or application file contains at least one drawing executed in color. Copies of this patent or patent application publication with color drawings will be provided by the Office upon request and payment of the necessary fee.

FIGS. 1A, 1B, 1C, and 1D illustrate the heme tethering principle. FIG. 1A illustrates the heme tethering principle. Heme iron acts as an electrophile and enables screening of fragments that tether to iron and block the gas tunnel. FIG. 1B shows the UV-Vis spectral change of the Soret band in Mb highlighting its sensitivity to ligand coordination events. FIGS. 1C and 1D show the gas tunnels in the crystal structure of horse heart myoglobin (PDB ID: 5ZZE, FIG. 1C) and M. tuberculosis DosS heme domain (PDB ID: 4YOF, FIG. 1D) as calculated by Caver Analyst 2.0 and visualized in PyMOL as a surface. Four tunnels were identified in the Mb tunnel calculation but only the top predicted tunnel is displayed. Only one gas tunnel was predicted for the DosS GAF-A domain structure. Residue side chains lining the tunnels are highlighted using stick representation in the same color as the tunnel surface. Overall tunnel position with respect to protein surface is visualized by inset.

FIGS. 2A, 2B, 2C and 2D show heme tether screening and characterization. FIG. 2A shows representative UV-Vis spectral shifts observed for various fragment types during screening. Most fragments show no shift in the Soret (left panel). Nitrile fragments (RCN) shift the Soret to 422 nm (middle panel) and phenyl hydrazine fragments (PhN₂H₄) shift the Soret to 432 nm. FIG. 2B shows the binding affinity (K_{d}) of various heme tethering nitriles obtained from the screen to ferric Mb. Inset shows the potential binding mechanism of nitriles to ferric heme. FIG. 2C shows the superposition of a [Fe^{III}(Por)(Im)(HT-N6)] DFT-minimized complex onto Mb crystal structures (PDB ID: 1J52 and 5IKS). The phenyl piperidine moiety of HT-N6 is able to enter Mb's gas tunnel as indicated by its superposition onto the Xe atom. HT-N6's hydroxyl can H-bond with Mb's backbone H64 carbonyl. FIG. 2D shows the binding affinity (K_{d}) of HT-N6 to heme proteins with different gas tunnel architectures, namely, cytochrome c, myoglobin (Mb) and DosS.

FIGS. 3A and 3B show the heme oxidation state specificity of nitriles. FIG. 1A shows the UV-Vis spectral transformation of ferric Mb upon titration of HT-N6. Spectral transformation was complete at 44 μM of HT-N6. FIG. 3B shows the titration of HT-N6 to ferrous Mb that demonstrates no change in spectra even when up to 20 mM of HT-N6 is added.

FIG. 4 shows the structures of the 20 fragment hits obtained from UV-Vis spectroscopy enabled screening of Mb. The fragments were classified as phenylhydrazines, nitriles and others.

FIG. 5 shows the UV-Vis spectra of fused heterocycle containing nitrile fragments titrated to ferric Mb. The chemical structures of the nitrile fragments are shown in each figure panel.

FIG. 6 shows the UV-Vis spectra showing the transition of Mb's Soret band and other visible feature when HT-N6 was titrated into ferric Mb. The starting ferric Mb spectra with 0 μM fragment HT-N6 is in brown and the ending spectra with 44 μM HT-N6 is in cyan.

FIG. 7 shows the UV-Vis spectral transformation observed for ferric Mb upon gradual addition of various nitrile fragments. Spectra shown are for HT-N1 to HT-N6 equilibrated with ferric Mb for 4-5 minutes in phosphate-buffered saline (137 mM NaCl, 2.7 mM KCl, 10 mM Na₂HPO₄, 1.8 mM KH₂PO₄, pH 7.4). The spectra shown is a difference spectra wherein absorbance of the ligand-free Mb has been subtracted. The addition of increasing amounts of nitrile fragments in Mb transitions the spectra from brown to orange to green to teal as has also been represented via arrows. Concentrations of proteins used for these experiments and the total starting/final amount of nitrile fragment added has been indicated in each figure panel.

FIG. 8 shows the binding affinity curves that were generated by plotting the sum of the maximal and minimal absorbance changes from the difference spectra in FIG. 7. The points were fitted using the quadratic binding equation on Origin to obtain the K_{d} and r² values reported.

FIG. 9 shows the UV-Vis spectra of the transition of Mb's Soret band and other visible feature when an analog of HT-N6 lacking the hydroxyl moiety was titrated into ferric Mb. The starting ferric Mb spectra with 0 μM fragment HT-N6-analog is in brown and the ending spectra with 469 μM HT-N6-analog is in teal.

FIG. 10A shows UV-Vis spectra of ferric horse heart cytochrotne c when HT-N6 is gradually added. The nitrile fragment HT-N6 was equilibrated with the protein for 4-5 minutes in phosphate-buffered saline (137 mM NaCl, 2.7 mM KCl, 10 mM Na₂HPO₄, 1.8 mM KH₂PO₄, pH 7.4).

FIG. 10B shows the difference spectra wherein absorbance of the ligand-free protein has been subtracted. Concentrations of proteins used for these experiments and the total starting/final amount of nitrile fragment added has been indicated.

FIG. 11A shows the UV-Vis spectral transformation observed for M. tuberculosis DosS GAF-A upon gradual addition of HT-N6. The nitrile fragment HT-N6 was equilibrated with the ferric heme protein for 4-5 minutes in phosphate-buffered saline (137 mM NaCl, 2.7 mM KCl, 10 mM Na₂HPO₄, 1.8 mM KH₂PO₄, pH 7.4). The spectra shown is a difference spectra wherein absorbance of the ligand-free protein has been subtracted. The addition of increasing amounts of HT-N6 in the protein transitions the spectra from brown to orange to green to teal as has also been represented via arrows. Concentrations of proteins used for these experiments and the total starting/final amount of nitrile fragment added has been indicated in each figure panel.

FIG. 11B shows the binding affinity curves generated by plotting the sum of the maximal and minimal absorbance changes from the difference spectra in FIG. 11A. The points were fitted using the quadratic binding equation on Origin to obtain the K_{d} and r² values reported.

FIG. 12 shows the UV-Vis spectra showing transition of heme Soret band and other visible feature when HT-N6 was titrated into various ferrous/ferrous-oxy heme proteins (myoglobin, hemoglobin and cytochrome c). The starting ferrous/ferrous-oxy protein spectra with 0 μM fragment HT-N6 is in brown and the ending spectra with 20 mM HT-N6 is in teal.

FIG. 13 shows that HT-N6 does not affect the growth of HEK-293T mammalian cells at concentrations as high at 100 micromolar

### DETAILED DESCRIPTION

Disclosed herein is a new small-molecule screening and ligand design approach called ‘heme-tethering’ to selectively inhibit metalloproteins including heme domains of therapeutically relevant enzymes. This approach includes the utilization of a UV-Vis spectroscopy-based primary screening method to identify fragments within chemically diverse libraries that target metalloproteins (e.g., heme proteins). This method has been used to identify a new class of nitrile-based metal-binding pharmacophores (MBPs) that bind only to ferric heme iron and can be made highly selective to the heme protein of interest by engineering its interactions with residues lining the protein's gas tunnel.

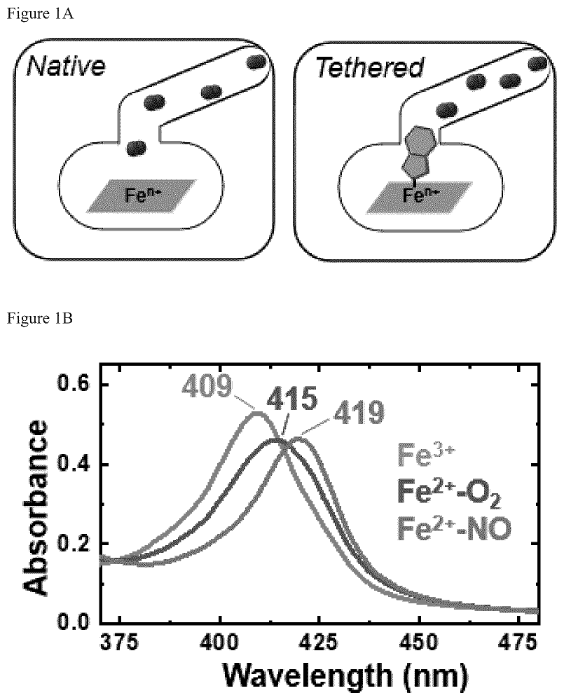

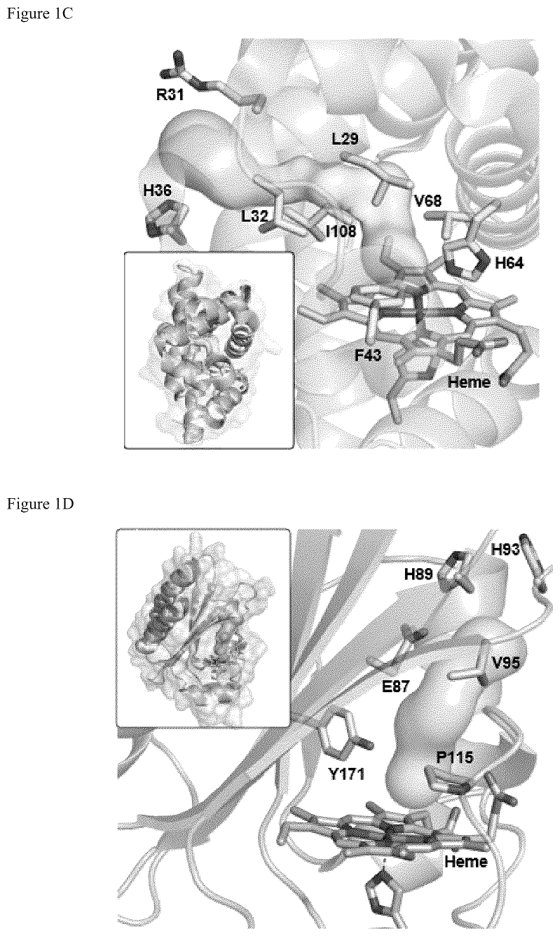

Described herein is a strategy using heme iron as the target electrophile (FIG. 1A), and UV-Vis spectroscopy as the screening method for fragment hits. UV-Vis spectroscopy offers a relatively straightforward and easily implementable tool to detect fragments/molecules that bind to protein's heme centre via bathochromic shifts in the heme Soret band. For instance, unligated ferric Mb has a Soret band peak at 409 nm, while ligated O₂- and NO-bound forms of Mb exhibit Soret band peaks at 416 nm and 419 nm, respectively (FIG. 1B). Similar shifts are anticipated from fragments that bind to heme proteins, which in turn can be used to identify hits. Fragment hits from these screens can be then optimized into potent therapeutics based on their interactions with heme-pocket amino acid residues of the target protein. Selectivity can be further enhanced by growing fragments into the interior gas tunnels of target heme proteins, such that they sterically block access to competing substrates (FIG. 1A). The topology of interior tunnels has been experimentally determined for several heme proteins including Mb (Tilton, R. F., et al., Biochemistry 23, 2849-2857, 1984), Hb (Lepeshkevich, S. V., et al., Biochim. Biophys. Acta 1864, 1110-1121, 2016) and bacterial enzyme H—NOX (Winter, M. B., et al., Proc. Natl. Acad. Sci. 108, E881-E889, 2011), and can also be computed from protein crystal structures using the CAVER program. Comparison of interior gas tunnel predictions of Mb and DosS (a mycobacterial heme sensor) reveal that Mb gas tunnel is lined with hydrophobic residues such as Leu, Ile, and Phe (FIG. 1C), while DosS's tunnel is highly hydrophilic and lined with polar residues such as His and Glu (FIG. 1D). Such differences in protein gas tunnel architecture can be further exploited to enhance selectivity of small-molecule heme tethers.

The following definitions are used, unless otherwise described: halo or halogen is fluoro, chloro, bromo, or iodo. Alkyl and alkoxy, etc. denote both straight and branched groups but reference to an individual radical such as propyl embraces only the straight chain radical (a branched chain isomer such as isopropyl being specifically referred to).

The term “(Cₐ-C_{b})alkyl” wherein a and b are integers refers to a straight or branched chain hydrocarbon alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

The term “heteroalkyl” refers to a straight or branched chain hydrocarbon alkyl radical, consisting of the stated number of carbon atoms and wherein from one to three carbons of the alkyl radical have been replaced with heteroatoms selected from the group consisting of O, N, and S, and wherein the nitrogen and sulfur atoms can optionally be oxidized and the nitrogen heteroatom can optionally be quaternized. The heteroatom(s) O, N and S can be placed at any interior position of the heteroalkyl group. Examples include —CH₂—CH₂—O—CH₃, —CH₂—CH₂—NH—CH₃, —CH₂—CH₂—N(CH₃)—CH₃, —CH₂—S—CH₂—CH₃, —S(O)—CH₃, and —CH₂—CH₂—S(O)₂—CH₃.

The term “alkenyl” refers to an unsaturated alkyl radical having one or more double bonds. Examples of such unsaturated alkyl groups ethenyl, 1- and 3-propenyl, 3-butenyl, and higher homologs and isomers.

The term “alkynyl” refers to an unsaturated alkyl radical having one or more triple bonds. Examples of such unsaturated alkyl groups ethynyl, 1- and 3-propynyl, 3-butynyl, and higher homologs and isomers.

The term “aryl” as used herein refers to a single aromatic ring or a multiple condensed ring system wherein the ring atoms are carbon. For example, an aryl group can have 6 to 10 carbon atoms, or 6 to 12 carbon atoms. Aryl includes a phenyl radical. Aryl also includes multiple condensed ring systems (e.g., ring systems comprising 2 rings) having about 9 to 12 carbon atoms or 9 to 10 carbon atoms in which at least one ring is aromatic. Such multiple condensed ring systems may be optionally substituted with one or more (e.g., 1 or 2) oxo groups on any cycloalkyl portion of the multiple condensed ring system. It is to be understood that the point of attachment of a multiple condensed ring system, as defined above, can be at any position of the ring system including an aryl or a cycloalkyl portion of the ring. Typical aryl groups include, but are not limited to, phenyl, indenyl, naphthyl, 1,2,3,4-tetrahydronaphthyl, anthracenyl, and the like.

The term “heteroaryl” as used herein refers to a single aromatic ring or a multiple condensed ring system. The term includes single aromatic rings of from about 1 to 6 carbon atoms and about 1-4 heteroatoms selected from the group consisting of oxygen, nitrogen and sulfur in the rings. The sulfur and nitrogen atoms may also be present in an oxidized form provided the ring is aromatic. Such rings include but are not limited to pyridyl, pyrimidinyl, oxazolyl or furyl. The term also includes multiple condensed ring systems (e.g. ring systems comprising 2 rings) wherein a heteroaryl group, as defined above, can be condensed with one or more heteroaryls (e.g., naphthyridinyl), heterocycles, (e.g., 1,2,3,4-tetrahydronaphthyridinyl), cycloalkyls (e.g., 5,6,7,8-tetrahydroquinolyl) or aryls (e.g. indazolyl) to form a multiple condensed ring system. Such multiple condensed ring systems may be optionally substituted with one or more (e.g., 1 or 2) oxo groups on the cycloalkyl or heterocycle portions of the condensed ring. In one embodiment a monocyclic or bicyclic heteroaryl has 5 to 10 ring atoms comprising 1 to 9 carbon atoms and 1 to 4 heteroatoms. It is to be understood that the point of attachment of a multiple condensed ring system (as defined above for a heteroaryl) can be at any position of the multiple condensed ring system including a heteroaryl, heterocycle, aryl or cycloalkyl portion of the multiple condensed ring system and at any suitable atom of the multiple condensed ring system including a carbon atom and heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, oxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl, benzofuranyl, benzimidazolyl and thianaphthenyl.

The term “heterocyclyl” or “heterocycle” as used herein refers to a single saturated or partially unsaturated ring or a multiple condensed ring system. The term includes single and bicyclic saturated or partially unsaturated rings from about 1 to 8 carbon atoms and from about 1 to 3 heteroatoms selected from the group consisting of oxygen, nitrogen and sulfur in the ring. The term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Such rings include but are not limited to azetidinyl, tetrahydrofuranyl or piperidinyl. It is to be understood that the point of attachment for a heterocycle can be at any suitable atom of the heterocycle. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl and tetrahydrothiopyranyl.

The term “alkoxy” refers to —O(alkyl) and the term “haloalkoxy” refers to an alkoxy that is substituted with one or more (e.g., 1, 2, 3, or 4) halo.

The term “haloalkyl” includes an alkyl group as defined herein that is substituted with one or more (e.g., 1, 2, 3, or 4) halo groups. One specific halo alkyl is a “(C₁-C₆)haloalkyl”.

The term cycloalkyl, carbocycle, or carbocyclyl includes saturated and partially unsaturated carbocyclic ring systems. In one embodiment the cycloalkyl is a monocyclic carbocyclic ring. Such cycloalkyls include “(C₃-C₇)carbocyclyl” and “(C₃-C₈)cycloalkyl”.

Specific values listed below for radicals, substituents, and ranges, are for illustration only; they do not exclude other defined values or other values Within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₃-C₈)cycloalkyl can be cyclopropyl, cyclobutyl, cyclopentyl, or cyclohexyl; (C₁-C₆)haloalkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; aryl can be phenyl, indenyl, or naphthyl; and heteroaryl can be furyl, imidazolyl, triazolyl, triazinyl, oxazolyl, isoxazolyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

### Metalloprotein Metal Moiety (i.e., Variable “X” of the Compounds of Formula I: X-L-Y)

The term “metalloprotein metal moiety” (i.e., variable “X” of the compounds of formula I) as used herein refers to a moiety (e.g., molecular fragment) that associates (e.g., is in contact) with a metal atom (e.g., central metal atom) of the metalloprotein. In one embodiment the metalloprotein metal moiety acts as a Lewis base by donating a pair of electrons to the central metal atom of a metalloprotein.

In one embodiment the metal moiety is a nitrile (i.e., —CN), wherein the nitrogen atom of the nitrile associates (e.g., is in contact) with a metal atom of the metalloprotein and the carbon atom of the nitrile is bonded to the remainder of the compound of formula I (i.e., the -L-Y fragment of the compounds of formula I).

In one embodiment the metal moiety is a —SCN or —OCN, wherein the nitrogen atom of the —SCN or —OCN moiety associates (e.g., is in contact) with a metal atom of the metalloprotein and the sulfur atom or oxygen atom of the —SCN or —OCN moiety is bonded to the remainder of the compound of formula I (i.e., the -L-Y fragment of the compounds of formula I).

In one embodiment the metal moiety is a —NCS, wherein the sulfur atom of the —NCS moiety associates (e.g., is in contact) with a metal atom of the metalloprotein and the nitrogen atom of the —NCS moiety is bonded to the remainder of the compound of formula I (i.e., the -L-Y fragment of the compounds of formula I).

In one embodiment the metalloprotein metal moiety is a hydrazine (i.e., —NHNH₂). When hydrazine is the metalloprotein metal moiety, the hydrazine moiety, after coming in contact with the metal in the protein (e.g., after reacting with the metal in the protein) is eliminated, rendering L-Y or Y (in the case wherein L is absent) of the formula I to associate (e.g., be in contact) with the metalloprotein.

### Linker

The term “linker” or variable L of the compounds of formula I as used herein refers to any group that connects the metalloprotein metal moiety (variable X of the compounds of formula I) to the gas tunnel binding moiety or substrate tunnel binding moiety (variable Y of the compounds of formula Y). The structural nature of the linker can be variable. It is to be understood that the linker (or portions thereof) may also bind to or be in proximity to the gas tunnel or substrate tunnel of the metalloprotein. For example, the fragment L-Y (or portions thereof) of the compounds of formula I may bind to the gas tunnel or substrate tunnel of the metalloprotein. It is also to be understood that in some embodiments the linker may be absent. For example, in some cases the metalloprotein metal moiety is directly bonded to the gas tunnel binding moiety or substrate tunnel binding moiety of the compounds of formula I.

In one embodiment L is (C₁-C₈)alkyl, (C₃-C₇)carbocyclyl, —(C₁-C₆)alkyl(C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl, wherein any (C₁-C₈)alkyl, (C₃-C₇)carbocyclyl, —(C₁-C₆)alkyl(C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of L is optionally substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment L is (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl, wherein any (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of L is optionally substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment L is (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl, wherein any (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of L is substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment L is (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl, wherein any (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of L is optionally substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment L is (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl, wherein any (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of L is substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment L comprises at least one of —OH, —SH, —NH₂, CO₂H, or a 4-7 membered heterocycle.

In one embodiment L comprises at least one of —OH or 4-7 membered heterocycle.

In one embodiment L comprises at least one of —OH, —OR^{L}, —SH, —SR^{L}, —NH₂, —NHR^{L}, —NR^{L}₂, —CO₂H, CO₂R^{L} or a 4-7 membered heterocycle.

In one embodiment L is (C₃-C₇)carbocyclyl, heterocyclyl, —(C₁-C₆)alkylheterocyclyl, or aryl, wherein any (C₃-C₇)carbocyclyl, heterocyclyl, —(C₁-C₆)alkylheterocyclyl, or aryl of L is optionally substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment L is heterocyclyl or —(C₁-C₆)alkylheterocyclyl, wherein any heterocyclyl or —(C₁-C₆)alkylheterocyclyl of L is substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment L is (C₃-C₇)carbocyclyl, heterocyclyl, —(C₁-C₆)alkylheterocyclyl, or aryl, wherein any (C₃-C₇)carbocyclyl, heterocyclyl, —(C₁-C₆)alkylheterocyclyl, or aryl of L is optionally substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment L is heterocyclyl or —(C₁-C₆)alkylheterocyclyl, wherein any heterocyclyl or —(C₁-C₆)alkylheterocyclyl of L is substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₇-C₁₂)alkyl, (C₁-C₁₂)heteroalkyl, or (C₃-C₇)carbocyclyl is optionally substituted with one or more halo.

In one embodiment L is heterocyclyl, wherein heterocyclyl is optionally substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or C₃-C₇carbocyclyl wherein the (C₁-C₁₂)alkyl, (C₁-C₁₂)heteroalkyl, or C₃-C₇carbocyclyl is optionally substituted with one or more halo.

In one embodiment L is heterocyclyl, wherein heterocyclyl is substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment L is:

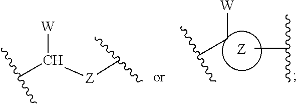

wherein;
W is a absent, H, —OH, —SH, —NH₂, CO₂H, or a 4-7 membered heterocycle; and
Z is a (C₃-C₇)carbocyclyl, aryl, or heterocycle, wherein the (C₃-C₇)carbocyclyl, aryl, or heterocycle is optionally substituted with one or more groups substituents independently selected from the one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment Z in the left hand structure of the above embodiment is absent.

In one embodiment L is:

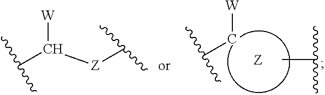

wherein;
W is a H, —OH, —SH, —NH₂, CO₂H, or a 4-7 membered heterocycle; and
Z is a heterocycle, wherein the heterocycle is optionally substituted with one or more groups substituents independently selected from the one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment Z in the left hand structure of the above embodiment is absent.

In one embodiment L is:

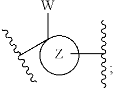

wherein;
W is a absent, H, —OH, —SH, —NH₂, CO₂H, or a 4-7 membered heterocycle; and
Z is a (C₃-C₇)carbocyclyl, aryl, or heterocycle.

In one embodiment L is:

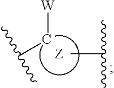

wherein;
W is a —OH, —SH, —NH₂, CO₂H, or a 4-7 membered heterocycle; and
Z is a heterocycle.

In one embodiment W is a —OH; and Z is a 3-7 membered monocyclic heterocycle.

In one embodiment L is:

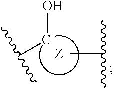

wherein Z is a heterocycle.

In one embodiment L is:

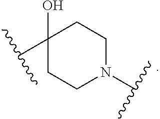 [SMILES: *N1CCC(*)(O)CC1]

In one embodiment L is:

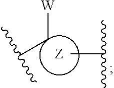

wherein;
W is a absent, H, —OH, —SH, —NH₂, CO₂H, or a 4-7 membered heterocycle; and
Z is a (C₃-C₇)carbocyclyl, aryl, or heterocycle, wherein the (C₃-C₇)carbocyclyl, aryl, or heterocycle is optionally substituted with one or more groups substituents independently selected from the one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment L is:

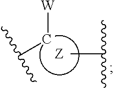

wherein;
W is a —OH, —SH, —NH₂, CO₂H, or a 4-7 membered heterocycle; and
Z is a heterocycle, wherein the heterocycle is optionally substituted with one or more groups substituents independently selected from the one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment W is a —OH; and Z is a 3-7 membered monocyclic heterocycle.

In one embodiment L is:

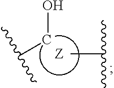

wherein Z is a heterocycle, wherein the heterocycle is optionally substituted with one or more groups substituents independently selected from the one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl, and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment L is:

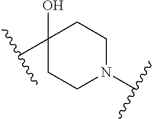 [SMILES: *N1CCC(*)(O)CC1]

wherein the hydroxypiperidine is optionally substituted with one or more groups substituents independently selected from the one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and

wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment L is absent.

### Gas Tunnel Binding Moiety or Substrate Tunnel Binding Moiety

The term “gas tunnel binding moiety or substrate tunnel binding moiety” as used herein is any molecular moiety that binds to the gas tunnel or substrate tunnel of the metalloprotein. The gas tunnel or substrate tunnel binding moiety generally increases the binding affinity of the compounds of formula I versus corresponding compounds that lack the gas tunnel or substrate tunnel binding moiety. The gas tunnel or substrate tunnel binding moiety can also provide selectivity of the compound of formula I for a particular metalloprotein (e.g., metalloenzyme) over one or more other proteins (e.g., metalloproteins such as metalloenzymes).

In one embodiment Y is (C₁-C₈)alkyl, (C₃-C₇)carbocyclyl, heterocyclyl, —(C₁-C₆)alkylheterocyclyl, aryl, —(C₁-C₆)alkylaryl, heteroaryl, —(C₁-C₆)alkylheteroaryl, CO₂H, CO₂R^{Z}, or —OR^{Z}, wherein any (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of Y is optionally substituted with one or more substituents independently selected from the group consisting of halo, —OH, —OR^{Y}, —SH, —SR^{Y}, NH₂, NHR^{Y}, —NR^{Y}₂, CO₂H, CO₂R^{Y}, (C₁-C₆)alkyl, (C₂-C₆)alkenyl, (C₂-C₆)alkynyl, 4-7 membered heterocyclyl, (C₃-C₇)carbocyclyl, and aryl, wherein (C₁-C₆)alkyl, (C₂-C₆)alkenyl, (C₂-C₆)alkynyl, 4-7 membered heterocyclyl, (C₃-C₇)carbocyclyl and aryl is optionally substituted with one or more halo or (C₁-C₄)alkyl;

wherein each R^{Y} is independently (C₁-C₁₂)alkyl, (C₁-C₁₂)heteroalkyl, or C₃-C₇carbocyclyl wherein the (C₁-C₁₂)alkyl, (C₁-C₁₂)heteroalkyl, or (C₃-C₇)carbocyclyl is optionally substituted with one or more halo; and

wherein each R^{Z} is independently (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl, wherein any (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of L is optionally substituted with one or more substituents independently selected from the group consisting of halo, —OH, —OR^{Y}, —SH, —SR^{Y}, NH₂, —NHR^{Y}, —NR^{Y}₂, CO₂H, CO₂R^{Y}, (C₁-C₆)alkyl, (C₂-C₆)alkenyl, (C₂-C₆)alkynyl, 4-7 membered heterocyclyl, (C₃-C₇)carbocyclyl, and aryl.

In one embodiment Y is (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl, wherein any (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of Y is optionally substituted with one or more substituents independently selected from the group consisting of halo, —OH, —OR^{Y}, —SH, —SR^{Y}, NH₂, NHR^{Y}, —NR^{Y}₂, CO₂H, CO₂R^{Y}, (C₁-C₆)alkyl, (C₂-C₆)alkenyl, (C₂-C₆)alkynyl, 4-7 membered heterocyclyl, (C₃-C₇)carbocyclyl, and aryl, wherein (C₁-C₆)alkyl, (C₂-C₆)alkenyl, (C₂-C₆)alkynyl, 4-7 membered heterocyclyl, (C₃-C₇)carbocyclyl and aryl is optionally substituted with one or more halo or (C₁-C₄)alkyl; and

wherein each R^{Y} is independently (C₁-C₁₂)alkyl, (C₁-C₁₂)heteroalkyl, or C₃-C₇carbocyclyl wherein the (C₁-C₁₂)alkyl, (C₁-C₁₂)heteroalkyl, or (C₃-C₇)carbocyclyl is optionally substituted with one or more halo.

In one embodiment Y is —(C₁-C₆)alkylaryl, wherein —(C₁-C₆)alkylaryl, is optionally substituted with one or more substituents independently selected from the group consisting of halo, —OH, —OR^{Y}, —SH, —SR^{Y}, NH₂, NHR^{Y}, —NR^{Y}₂, CO₂H, CO₂R^{Y}, (C₁-C₆)alkyl, (C₂-C₆)alkenyl, (C₂-C₆)alkynyl, 4-7 membered heterocyclyl, (C₃-C₇)carbocyclyl, and aryl, wherein (C₁-C₆)alkyl, (C₂-C₆)alkenyl, (C₂-C₆)alkynyl, 4-7 membered heterocyclyl, C₃-C₇carbocyclyl and aryl is optionally substituted with one or more halo or (C₁-C₄)alkyl; and

wherein each R^{Y} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted With one or more halo.

In one embodiment Y is —(C₁-C₆)alkylaryl, wherein —(C₁-C₆)alkylaryl, is optionally substituted with one or more substituents independently selected from the group consisting of halo, —OH, —OR^{Y}, —SH, —SR^{Y}, NH₂, NHR^{Y}, —NR^{Y}₂, CO₂H, CO₂R^{Y}, (C₁-C₆)alkyl, (C₂-C₆)alkenyl, (C₂-C₆)alkynyl, 4-7 membered heterocyclyl, (C₃-C₇)carbocyclyl, and aryl, wherein (C₁-C₆)alkyl, (C₂-C₆)alkenyl, (C₂-C₆)alkynyl, 4-7 membered heterocyclyl, (C₃-C₇)carbocyclyl and aryl is optionally substituted with one or more halo or (C₁-C₄)alkyl; and

wherein each R^{Y} is independently (C₁-C₁₂)alkyl, (C₁-C₁₂)heteroalkyl, (C₃-C₇)carbocyclyl wherein the (C₁-C₁₂)alkyl, (C₁-C₁₂)heteroalkyl, or (C₃-C₇)carbocyclyl is optionally substituted with one or more halo.

In one embodiment Y is benzyl, wherein benzyl, is optionally substituted with one or more substituents independently selected from the group consisting of halo, —OH, —OR^{Y}, —SH, —SR^{Y}, NH₂, NHR^{Y}, —NR^{Y}₂, CO₂H, CO₂R^{Y}, (C₁-C₆)alkyl, (C₂-C₆)alkenyl, (C₂-C₆)alkynyl; and

wherein each R^{Y} is independently (C₁-C₁₂)alkyl.

In one embodiment Y is benzyl.

In one embodiment Y is (C₁-C₈)alkyl, (C₃-C₇)carbocyclyl, heterocyclyl, —(C₁-C₆)alkylheterocyclyl, aryl, —(C₃-C₆)alkylaryl, heteroaryl, or —(C₁-C₆)alkylheteroaryl, wherein any (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of Y is optionally substituted with one or more substituents independently selected from the group consisting of halo, CN, —OH, —OR^{Y}, —SH, —SR^{Y}, NH₂, NHR^{Y}, —NR^{Y}₂, CO₂H, CO₂R^{Y}, and R¹, wherein R¹ is selected from (C₁-C₆)alkyl, (C₂-C₆)alkenyl, (C₂-C₆)alkynyl, C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl,

wherein any (C₁-C₆)alkyl, (C₂-C₆)alkenyl, (C₂-C₆)alkynyl, C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of R¹ is optionally substituted with one or more substituents independently selected from the group consisting of halo, CN, —OH, —OR^{Y}, —SH, —SR^{Y}, NH₂, NHR^{Y}, —NR^{Y}₂, CO₂H, CO₂R^{Y}, or (C₁-C₄)alkyl;, and

wherein each R^{Y} is independently (C₁-C₁₂)alkyl, (C₁-C₁₂)heteroalkyl, or C₃-C₇carbocyclyl wherein the (C₁-C₁₂)alkyl, (C₁-C₁₂)heteroalkyl, or (C₃-C₇)carbocyclyl is optionally substituted with one or more halo.

In one embodiment Y is hydrogen.

One embodiment provides a novel compound of formula I:

X-L-Y I

or a salt thereof wherein:
X is nitrile (—CN), hydrazine, —SCN, —NCS, or —OCN;
L is absent, (C₁-C₈)alkyl, (C₃-C₇)carbocyclyl, —(C₁-C₆)alkyl(C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl, wherein any (C₁-C₈)alkyl, (C₃-C₇)carbocyclyl, —(C₁-C₆)alkyl(C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of L is optionally substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl;

each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo; and

Y is (C₁-C₈)alkyl, (C₃-C₇)carbocyclyl, heterocyclyl, —(C₁-C₆)alkylheterocyclyl, aryl, (C₁-C₆)alkylaryl, heteroaryl, —(C₁-C₆)alkylheteroaryl, CO₂H, CO₂R^{Z}, or —OR^{Z}, wherein any (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of Y is optionally substituted with one or more substituents independently selected from the group consisting of halo, —OH, —OR^{Y}, —SH, —SR^{Y}, NH₂, NHR^{Y}, —NR^{Y}₂, CO₂H, CO₂R^{Y}, (C₁-C₆)alkyl, (C₂-C₆)alkenyl, (C₂-C₆)alkynyl, 4-7 membered heterocyclyl, (C₃-C₇)carbocyclyl, and aryl, wherein (C₁-C₆)alkyl, (C₂-C₆)alkenyl, (C₂-C₆)alkynyl, 4-7 membered heterocyclyl, (C₃-C₇)carbocyclyl and aryl is optionally substituted with one or more halo or (C₁-C₄)alkyl;

each R^{Y}is independently (C₁-C₁₂)alkyl, (C₁-C₁₂)heteroalkyl, or C₃-C₇carbocyclyl wherein the (C₁-C₁₂)alkyl, (C₁-C₁₂)heteroalkyl, or (C₃-C₇)carbocyclyl is optionally substituted with one or more halo; and

each R^{Z} is independently (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl, wherein any (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of L is optionally substituted with one or more substituents independently selected from the group consisting of halo, —OH, —OR^{Y}, —SH, —SR^{Y}, NH₂, NHR^{Y}, —NR^{Y}₂, CO₂H, CO₂R^{Y}, (C₁-C₆)alkyl, (C₂-C₆)alkenyl, (C₂-C₆)alkynyl, 4-7 membered heterocyclyl, (C₃-C₇)carbocyclyl, and aryl.

One embodiment provides a novel compound of the embodiment directly above wherein X, L, and Y are as defined in any other embodiment provided herein.

### Metalloprotein

Metalloproteins include proteins that comprise one or more metal atoms. In one embodiment at least one of the metal atoms of the metalloprotein is a “central metal atom”. The term “central metal atom” as used herein means the metal that is key for structural stability and/or function of the protein. In one embodiment the metalloprotein is a metalloenzyme. In one embodiment the metalloprotein is a heme enzyme. In one embodiment the metalloprotein is an metalloenzyme comprising an iron atom (e.g., a central iron atom). In one embodiment the metalloprotein is an metalloenzyme comprising an iron atom (e.g., a central iron atom) wherein the iron atom is in the Fe³⁺ oxidation state.

### Gas Tunnel or Substrate Tunnel

Gas tunnel or substrate tunnels are interior, hollow spaces in a protein that enable entry of gases or substrates to the active site. These are dynamic in nature and open up/close down based on protein's function. FIG. 1C and FIG. 1D represent such gas/substrate tunnels for two proteins, namely Mb and DosS.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention.

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “mammal” as used herein refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient. In one embodiment, the patient is a human patient.

The compositions including pharmaceutical compositions described herein can comprise one or more excipients. When used in combination with the compositions (e.g., pharmaceutical compositions) described herein the term “excipients” refers generally to an additional ingredient that is combined the composition (e.g., pharmaceutical compositions) described herein to provide a corresponding composition. For example, when used in combination with the compositions (e.g., pharmaceutical compositions) described herein the term “excipients” includes, but is not limited to: carriers, binders, disintegrating agents, lubricants, sweetening agents, flavoring agents, coatings, preservatives, and dyes.

The compositions (e.g., pharmaceutical compositions) described herein can be administered to a mammalian host, such as a human patient in a variety of forms adapted to the chosen route of administration, i.e., orally or parenterally, by intravenous, intramuscular, topical or subcutaneous routes or intratumorally. For oral administration the compositions (e.g., pharmaceutical compositions) can be formulated as a solid dosage form with or without an enteric coating.

Thus, the present compositions (e.g., pharmaceutical compositions) may be systemically administered, e.g., orally, in combination with a pharmaceutically acceptable vehicle such as an inert diluent, excipient or an assimilable edible carrier. They may be enclosed in hard or soft shell gelatin capsules, may be compressed into tablets, or may be incorporated directly with the food of the patient's diet. For oral therapeutic administration, compositions (e.g., pharmaceutical compositions) may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 90% of the weight of a given unit dosage form. The amount of active agents in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations, particles, and devices.

The compositions (e.g., pharmaceutical compositions) may also be administered intravenously or intramuscularly by infusion or injection. Solutions of the compositions (e.g., pharmaceutical compositions) can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the compositions (e.g., pharmaceutical compositions) described herein in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the compositions (e.g., pharmaceutical compositions) may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina, nanoparticles, and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compositions (e.g., pharmaceutical compositions) can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compositions (e.g., pharmaceutical compositions) and agents thereof required for use in treatment will vary with the route of administration, the nature of the condition being treated and the age and condition of the patient and will be ultimately at the discretion of the attendant physician or clinician.

In general, however, a suitable dose will be in the range of from about 1 to about 500 mg/kg, e.g., from about 5 to about 400 mg/kg of body weight per day, such as 1 to about 250 mg per kilogram body weight of the recipient per day.

The compound is conveniently formulated in unit dosage form; for example, containing 5 to 500 mg, 10 to 400 mg, or 5 to 100 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The invention will now be illustrated by the following non-limiting Example.

### EXAMPLE 1

### Methods

All chemicals, unless otherwise specified, were obtained from Millipore Sigma (St. Louis. MO), Tokyo Chemical Industry (Tokyo, Japan), TCI America (Portland, OR), Oakwood Chemical (Estill, SC), VWR International (Radnor, PA), Neta Scientific (Hainesport, NJ), or Fisher Scientific (Hampton, NH).

### Expression and Purification of DosS

DosS plasmid DNA was designed by inserting the DosS GAF-A (heme domain of DosS) gene in a pET23a+ vector with a N-terminal histidine tag and synthesized from Genscript. DosS plasmid was sequenced at the UMN Genomics Centre by Sanger method. DosS was expressed and purified from BL21 E. coil cells via over-expression and His-tag affinity purification using methods described previously (Sanyal, R., et al., J. Biol. Inorg. Chem. 25, 181-186, 2020).

Ten primary cultures tubes were prepared by mixing into each sterile culture tubes: 5 mL of 2XYT liquid medium previously autoclaved, 5 μL of 100 mg/mL ampicillin, 5 μL of 37 mg/mL chloramphenicol, and one single colony of E. coli BL21(DE3) gold competent cells previously transformed with DosS (GAF-A domain) and GroES/EL plasmids. Cultures were left to grow overnight for approximately 14 h at 37° C. and 200 rpm. Four secondary cultures were prepared using four conical flasks containing 1.5 L of 2XYT liquid medium previously autoclaved. To each of these, 1.5 mL of 100 mg/mL ampicillin, 1.5 mL of 37 mg/mL chloramphenicol, and 10 mL of primary cultures were added. Cultures were left to grow at 37° C. and 200 rpm until they reached an OD₆₀₀ of 0.4-0.6. After this, the temperature was lowered to 22° C. and speed lowered to 175 rpm. Then, 45 mg of heme chloride dissolved in 4.5 mL of 0.1 N NaOH, were added to each of the culture flasks. The cultures were shaken until they reached an OD₆₀₀ of approximate 0.8. Protein expression was induced by adding 1.5 mL of 1 M isopropyl-β-D-thiogalactoside (IPTG) to each culture flask. Cultures were induced for 24 h at 18° C. and 160 rpm. Cells were finally harvested at 8000 rpm, 30 min, and 4° C. The supernatant was discarded, and cell pellets were collected into falcon tubes, frozen and stored at −20° C. until lysis.

The cell pellet (of approximate 25 g) was thawed and 100 mL of lysis buffer (50 mM NaH₂PO₄, 250 mM NaCl, 10% glycerol, 1% Triton-X, one Pierce™ protease inhibitor tablet, Thermo Fisher Scientific) was added to it. The mixture was shaken at 30° C. for approximate 30-40 min. The cell lysate was, then, sonicated for 20 min (amplitude: 60%, 30 sec on, 30 sec off) and centrifuged for 30 min at 4° C. and 20,000 rpm. The red colored supernatant was filtered, yielding approximately 100 mL of protein containing sample. Using a GE Äkta start system, a 5 mL GE HisTrap™ FF column was initially equilibrated with 5 column volumes (CV) of Buffer A (50 mM NaH₂PO₄, 20 mM imidazole, 500 mM NaCl, 10% glycerol, pH 7.5) at 5 mL/min. The sample was loaded at 1 mL/min, and any unbound sample was washed with 5 CV of Buffer A at 2 mL/min. Sample was eluted in the following manner: i) linear gradient: 0% to 20% Buffer B (50 mM NaH₂PO₄, 400 mM imidazole, 500 mM NaCl, 10% glycerol, pH 7.5) over 5 CV, ii) isocratic step 20% Buffer B over 10 CV (or until baseline is flattened), iii) isocratic step: 100% Buffer B over 7 CV. The fraction size was set to 2 mL and a final equilibration with 5 CV of Buffer A was performed to conclude the method. Fractions were collected upon analysis of their UV-Vis spectra, in which a Soret peak (ferric DosS GAF-A=406 nm) was present and prominent, as well as r/z (406/280) value, preferably higher than 4.0. The collected fractions were poured into a Spectra/Por™ dialysis membrane (MWCO: 6-8 kDa) and dialyzed overnight (approximately 18 h) in 50 mM Tris-HCl, 300 mM NaCl, pH 7.5. Finally, the dialyzed sample was concentrated to approximately 10 mL. The collected fractions were pooled together and concentrated to approximately 500 μL. Finally, glycerol was added and protein aliquots were flash frozen in liquid nitrogen and stored at −80° C.

### Pyridine Hemochromagen Assay

The extinction coefficients for the Soret bands of commercial horse heart myoglobin (409 nm), hhMb (Sigma: M1882) and recombinant DosS (GAF-domain, 406 nm) were determined according to the protocol reported by Barr and Guo (Barr, I., et at, Bio Protoc 5, 1-8 (2015).) and found to be 165.7±2.4 mM⁻¹ cm⁻¹ and 161.3±7.7 mM⁻¹ cm⁻¹, respectively.

### Fragment-Based Screening by Heme-Absorbance Shift Assay

Fragment libraries Maybridge1000 and Maybridge1895 (Maybridge/Fisher Scientific) were obtained from the High-Throughput Screening Laboratory (HTSL) at the Institute for Therapeutics Discovery and Development (ITDD) of the University of Minnesota-Twins Cities. The two libraries share ˜70% similarity in fragments. Using the LabCyte ECHO Liquid Handler (HTSL-ITDD), 500 nL of fragments were dispensed to Greiner Bio-One 384-well microtiter plates into columns 3 to 22, reserving columns 1 and 24, and 2 and 23 for blank and controls, respectively. The final fragment concentration in each well is 1 mM. Plates were sealed and stored in −20° C. Each plate was thawed at least 30 minutes before each absorbance assay, and then it was unsealed. Assay buffer (PBS, 1% DMSO, 0.01% Triton-X, pH 7.4) was added to each well according to its location on the plate (84.5 μL for columns 3 to 22, 85 μL for columns 2 and 23, and 100 μL for columns 1 and 24). The plate was centrifuged (1 min, 1000 rpm, rt), shaken (10 min) on a Tecan Spark plate reader, and then the full UV-Vis spectrum from 200-700 nm was recorded for the whole plate. 5 μL of 100 μM horse heart wild-type myoglobin (Sigma: M1882) were added to each well, except for columns 1 and 24. Then, the plate was centrifuged again (1 min, 1000 rpm, rt), shaken (10 min) on a Tecan Spark plate reader, and then incubated and shaken for 2 hrs on the plate shaker. After incubation of the fragments with ferric Mb, the full UV-Vis spectrum from 200-700 nm was recorded for the whole plate. Next, 5 μL of 20 mM TMPD/200 mM sodium ascorbate solution were added to each well, except for columns 1 and 24. Subsequently, 5 μL of 500 μM PROLI-NONOate solution (in 0.01 M NaOH) were added to each well, except for columns 1 and 24. The plate was centrifuged again (1 min, 1000 rpm, rt) and shaken (10 min) on a Tecan Spark plate reader. Finally, the full UV-Vis spectrum from 200-700 nm was recorded for the whole plate once more. UV-Vis data obtained from these measurements were analysed to determine which fragments shift the Soret peak position. The ones that shifted peak position >10 nm were considered ‘hits’ and analysed further.

### UV-Vis Spectrophotometric Ligand Affinity Studies

UV-Vis spectrophotometric ligand affinity studies for ferric and ferrous-oxy heme proteins were performed using a Cary4000 UV-Vis spectrophotometer. These studies were performed in an aerobic environment. Lyophilized horse heart Mb, human Hb, equine heart cytochrome c were in their ferric form when dissolved in buffer. DosS GAF-A was purified in its ferric form. Ferrous-oxy forms of Mb and hemoglobin (Hb) were prepared by addition of a small aliquot of sodium dithionite to the proteins (in air) and running the protein through PD-10 columns twice to remove excess dithionite/other products. Once the ferrous-oxy form/ferric form of the proteins were confirmed via UV-Vis spectroscopy, increasing aliquots of fragments were added to the protein solution. For these, stock solutions of the fragment hits (typically 200-500 mM, in DMSO) were used to prepare a 2-fold serial dilution going down to sub-millimolar concentrations (in DMSO). These serial dilution stock solutions were then used to pipette aliquots (typically 2.5 but higher if necessary) of the appropriate solution in order to result in a concentration curve (typically 1 μM to 5 mM, but adjustable to fragment affinity and solubility). The protein concentration was set to ˜1 μM to obtain a desirable absorbance reading at the Soret peak region. Between each addition of ligand, there was an equilibration time (4-5 min). The initial volume of the cuvette was 2.5 mL and the buffer used was phosphate-buffered saline (137 mM NaCl, 2.7 mM KCl, 10 mM Na₂HPO₄, 1.8 mM KH₂PO₄, pH 7.4). Increments of fragment are added to a cuvette containing ˜1 μM heme protein in one single experiment, without washing the cuvette between points. This method was employed for those compounds that do not present intrinsic absorbance in the 350-500 nm region. For those fragments with some intrinsic absorbance in the aforementioned region, a second titration was performed with the same conditions, but without protein. This latter data was subtracted from the titration with protein before data analysis.

UV-Vis spectrophotometric ligand affinity studies for ferrous heme proteins were performed using a Cary 60 UV-Vis spectrophotometer. These studies were performed in an anaerobic environment maintained by Coy glovebag. All buffers and DMSO used for these studies were degassed in a Schlenk line set-up and equilibrated in glovebag's anaerobic atmosphere for more than 48 hrs. Ferrous form of Mb and Hb was prepared by addition of a small aliquot of sodium dithionite to the proteins in the glovebag and running the protein through PD-10 column to remove excess dithionite/other products. Ferrous Hb and Mb were then subjected to similar fragment binding studies as above except in an anaerobic environment. From acquired spectral data, the difference spectra was generated by subtracting the ligand-free spectrum from each concentration point spectrum. The sum of the maximal and minimal differences in absorbance was plotted for each concentration point for each ligand. Data was fitted on Prism 9 (GraphPad Software, San Diego, CA) or Origin 8 using the quadratic binding equation (Eq. 1) (Gee, C. T. et al., Nat. Protoc. 11, 1414-1427, 2016).

$$Y = A \times \frac{( {K}_{d} + L + P ) - \sqrt{{( {K}_{d} + L + P )}^{2} - 4 PL}}{2 P} \qquad Eq . 1$$

where Y is the observed change in absorbance, A is the maximum change in absorbance, K_{d} is the dissociation constant of the ligand, L is the ligand concentration and P is the protein concentration. The maximal change in absorbance, A, is obtained through nonlinear regression. The errors were obtained by calculating the standard deviation of K_{d} values in individual measurements. In case this error was lower than the error of Quadratic binding equation fitting, we report the error from fitting. We have reported as many significant figures as are consistent with the estimated error.

### Tunnel Calculation Studies

Protein tunnels in crystal structures of DosS (PDB ID: 4YOF) and Mb (PDB ID: 5ZZE) were identified by Caver Analyst 2.0 (Jurcik, A. et al., Bioinformatics 34, 3586-3588, 2018). The heme was chosen as the tunnel starting point in both proteins and, in addition to the protein structure, the heme was included as a residue in tunnel calculation while all other molecules in the .pdb files were excluded. DosS tunnel analysis utilized a probe radius of 0.9 Å, a shell radius of 3 Å, and shell depth of 4 Å while Mb tunnel analysis utilized a probe radius of 0.9 Å, a shell radius of 3 Å, and shell depth of 5.5 Å. Clustering threshold was kept at 3.5 for both calculations and tunnels were approximated by 20 spheres. FIGS. 1C and 1D show the gas tunnel in crystal structures of DosS and Mb proteins that were created using PyMOL (The PyMOL Molecular Graphics System, Schrödinger, LLC).

### Results and Discussion

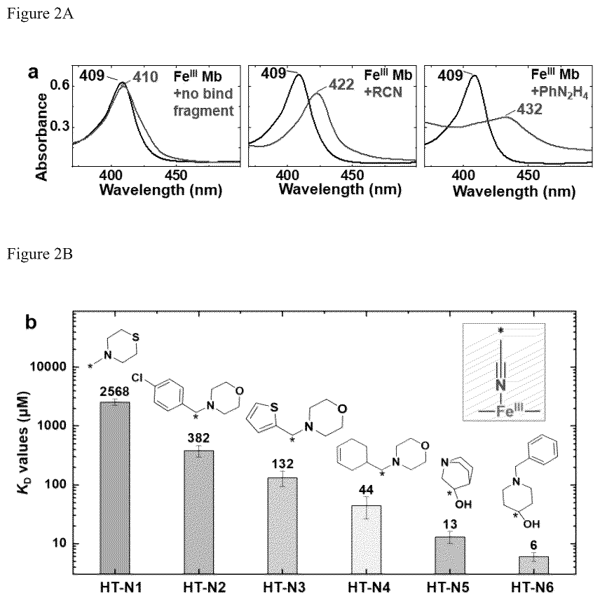

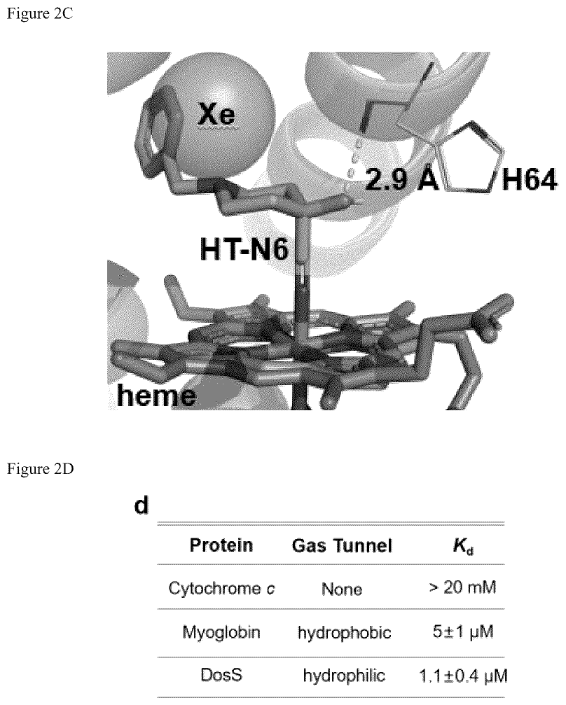

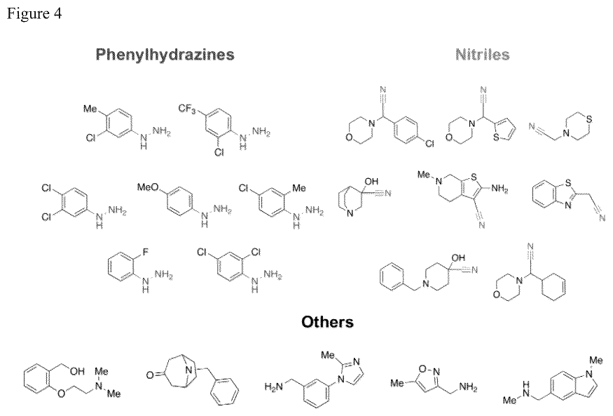

Screening began for fragment hits that tether to heme using horse heart Mb as a model heme protein. Mb has been the subject of intensive structure-function studies, and the topology of internal gas tunnels through which various ligands (O₂/NO/CO) enter its heme pocket are well characterized (Sherwood, C., et al., J. Mol. Biol. 193, 227, 1987; Maurus, R. et al., Biochim. Biophys. Acta-Protein Struct. Mol. Enzymol. 1341, 1-13, 1997; Kitahara, M., et al., Cryst. Growth Des. 19, 6136-6140. 2019; Tomita, A., et al., J. Exp. Biol. 213, 2748-2754, 2010). Furthermore, as a commercially available and inexpensive heme protein, Mb is a viable choice to run extensive screening experiments. Two pharmacophore-rich, rule-of-three compliant fragment libraries (Maybridge 1000 and Maybridge 1895) comprising of 2895 compounds with diverse chemical and structural attributes were screened for tethering hits. More specifically, ferric Mb was incubated with different fragments using a microtiter 384-well plate assay format, and the UV-Vis absorption spectra (200-700 nm) was acquired using a Tecan Spark plate reader (see Methods for details). Incubation with most fragments (n=2975) did not exhibit any shift in the heme Soret peak compared to that of unligated ferric Mb (left panel, FIG. 2A). Twenty fragment hits were identified (FIG. 4) based on the criterion that heme Soret shifts of greater than 10 nm was observed on incubating them with Mb. Moreover, two functional groups were widely prevalent in these hits, namely nitrile (8 fragments with representative spectra FIG. 2A, middle panel) and phenylhydrazine (7 fragments with representative spectra FIG. 2A, right panel). Overall, using a relatively straightforward and efficient absorbance assay, twenty fragment-based heme tethers were identified from a total of 2895 compounds corresponding to a hit rate of 0.6%.

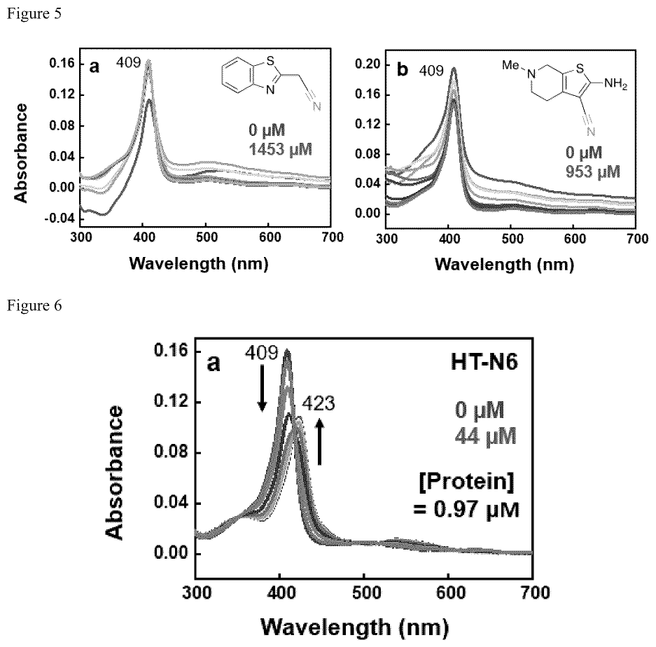

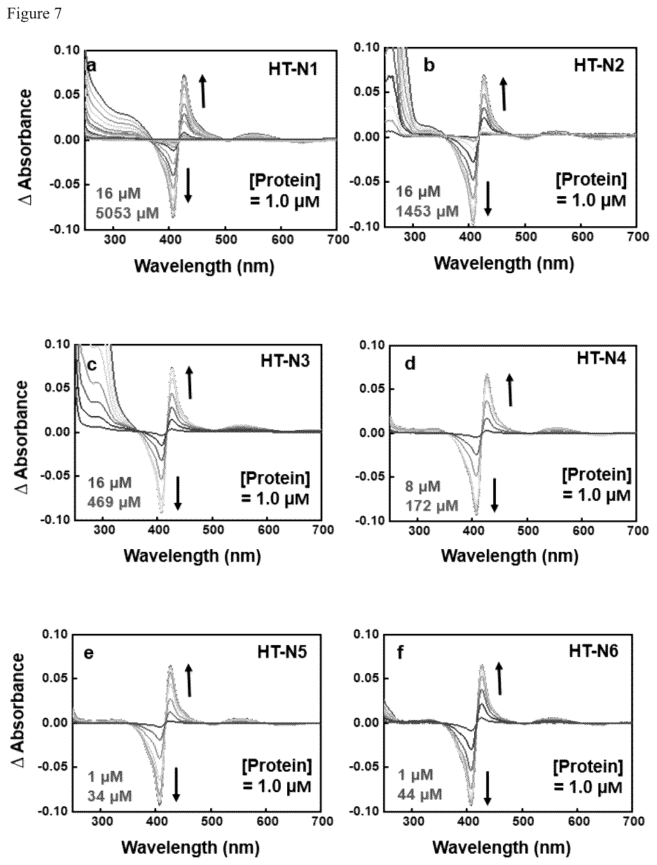

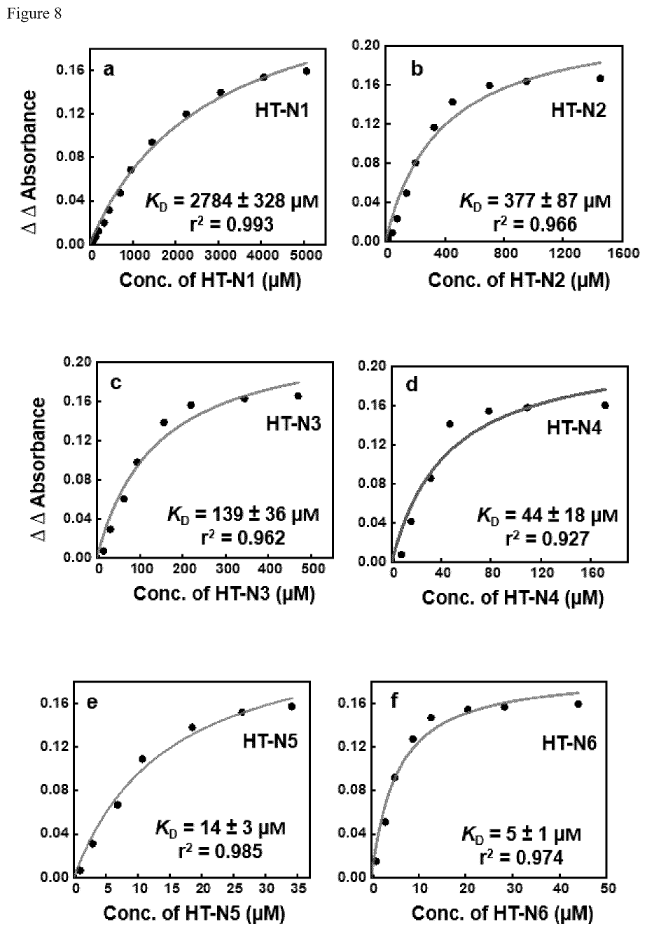

The covalent binding of phenylhydrazine to heme has been explored previously (Kunze, K. L., et al., J. Am. Chem. Soc. 105, 1380-1381, 1983; Ringe, D., et al., Biochemistry 23, 2-4, 1984; Ortiz de Montellano, P. R., Biochimie 77, 581-593, 1995; de Montellano, P. R. O., et al., Biochemistry 24, 1147-1152, 1985; Augusto, O., et al., J. Biol. Chem. 257, 6231-6241, 1982; Goldberg, B., et al., Mol. Pharmacol. 13, 832-839, 1977; Saito, S. & Itano, H. A. beta-meso-Phenylbiliverdin IX alpha and N-phenylprotoporphyrin IX, products of the reaction of phenylhydrazine with oxyhemoproteins. 78, 5508-5512, 1981; Wang, B., et al., J. Inorg. Biochem. 164, 1-4, 2016). Despite decades of intensive research on Mb, no studies heretofore have reported the nitrile group as a stable ligand to ferric heme. To better understand the chemical and structural attributes of nitrile tethers that impact their binding to ferric Mb, UV-Vis spectrophotometric titrations were conducted to determine the dissociation constant (K_{d}) of various nitrile hits (FIG. 2B). Notably, nitriles attached to fused heterocycles did not exhibit any binding even at millimolar ligand concentrations (FIG. 5). Monocyclic or non-fused bicyclic nitriles, on the other hand, when added in increasing concentrations (nM to mM) exhibited varying degrees of affinity to heme, as indicated by the gradual shift of heme Soret band peak from 409 nm to 422 nm (FIGS. 6, 7, and 8). The thiomorpholine N-carbonitrile fragment (HT-N1) binds to heme with a rather high K_{d} value of 2568±328 μM. Substituting the C2-methylene group to a more aromatic-rich and hydrophobic branched p-Cl-phenyl (HT-N2) enhances heme binding affinity nearly ten-fold (K_{d}=382±87 μM), likely due to a better fit within the heme pocket. Incorporation of a smaller thiophene to N-methyl morpholine (HT-N3, K_{d}=132±39 μM) results in approximate three-fold enhancement of heme binding affinity, and highlights a systematic enhancement of heme tethering capability of nitriles with branched groups. Furthermore, moving from planar p-Cl-phenyl and thiophene groups to flexible cyclohexene (HT-N4) moiety results in an overall three-fold enhancement of heme binding affinity (K_{d}=44±18 μM). These studies suggest that the flexibility of branching moieties is important and likely help with positioning nitrile tethers in the heme pocket. Moving to morpholine-lacking moieties with a piperidine skeleton and a cyanohydrin group further enhances the binding affinity in HT-N5 (K_{d}=13±3 μM) and HT-N6 (K_{d}=6±1 μM). Ultimately, the observed trends in binding affinities ranging over three-orders of magnitude highlight the important role of branching, branch flexibility and nature of branching moieties towards heme binding capability of nitrile tethers.

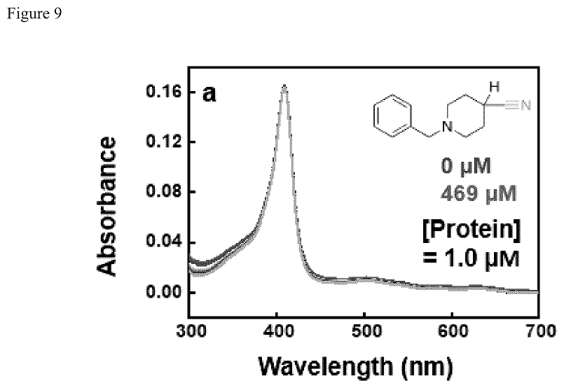

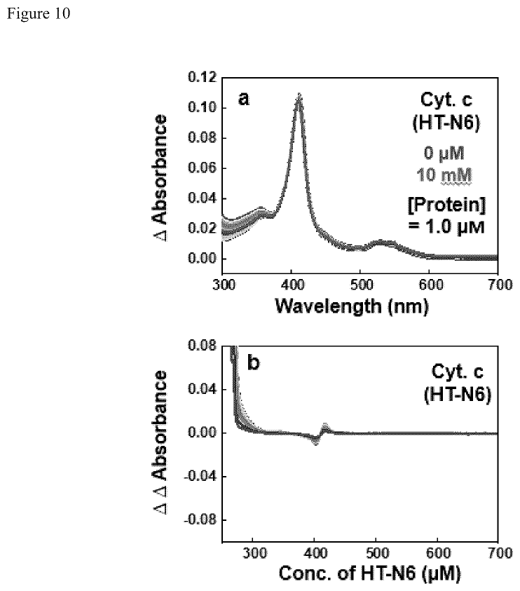

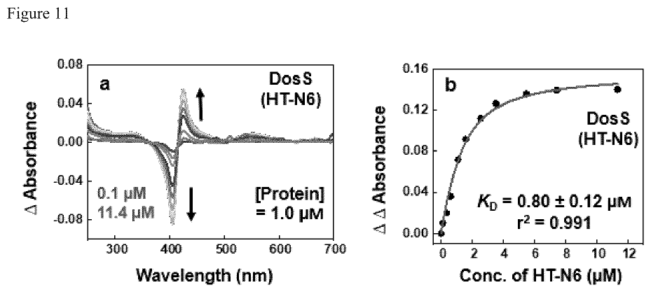

In order to visualize how the nature of branching moieties can impact nitrile tethering to Mb heme, a density functional theory (DFT) minimized structure of porphyrin-imidazole-bound HT-N6 (the most potent tether from screening studies) was superimposed with the Xe co-crystallized structure of Mb (FIG. 2C). In addition to residues lining the Mb heme pocket, the Xe co-crystallized structure also reveals interior gas tunnels. The superposition suggests that the hydroxyl group of HT-N6 can form a hydrogen-bond with the H64 backbone carbonyl residue in Mb heme pocket and thereby enhance binding affinity. Such a hypothesis is further supported by affinity studies on hydroxyl-lacking version of HT-N6 which does not bind to Mb heme even at concentrations that are hundred-fold higher than the K_{d} of HT-N6 (FIG. 9). Beyond hydrogen-bonding, the hydroxyl group of HT-N6 may also be responsible for directing the N-benzyl piperidine moiety to a location that sterically blocks the gas tunnel and limits competing substrates from entering the Mb heme pocket. Note that the N-benzyl piperidine moiety overlays on Xe atom located at the opening of gas tunnel of Xe co-crystallized Mb structures (FIG. 2C). Such a gas tunnel blocking moiety is absent in HT-N5 which has a three-fold lower binding affinity as compared to HT-N6. To better understand how the structural and chemical topologies of interior gas tunnels impact small molecule interaction, the binding of HT-N6 to other ferric heme proteins including cytochrome c (mammalian electron transfer protein) and DosS (hypoxia sensing/signalling enzyme critical for regulating mycobacterial dormancy) was also studied. The absence of any gas tunnel in cytochrome c makes it a good negative control to probe role of gas tunnels in small-molecule heme tethering. As anticipated, even at high millimolar concentrations of HT-N6, no binding to cytochrome c (FIG. 2D, FIG. 10) was observed. On the other hand, more than five-fold enhanced binding of HT-N6 (K_{d}=1.1±0.4 μM, FIG. 2D, FIG. 11) to DosS as compared to Mb (K_{d}=6±1 μM) was noted. The gas tunnel of DosS (FIG. 1D) is lined by hydrophilic amino acid residues including Y171 and E87 that are strong H-bond donors/acceptors as compared to the gas tunnel of Mb which is lined by hydrophobic residues (FIG. 1C). The higher binding affinity of HT-N6 to DosS likely arises from the hydrophilic hydroxyl and tertiary amine moieties of HT-N6 which will prefer hydrophilic gas tunnels of DosS. Overall, these studies reveal that topological features including the structure and chemistry of heme pocket and interior gas tunnels can play a crucial role towards binding affinity of nitrile tethers.

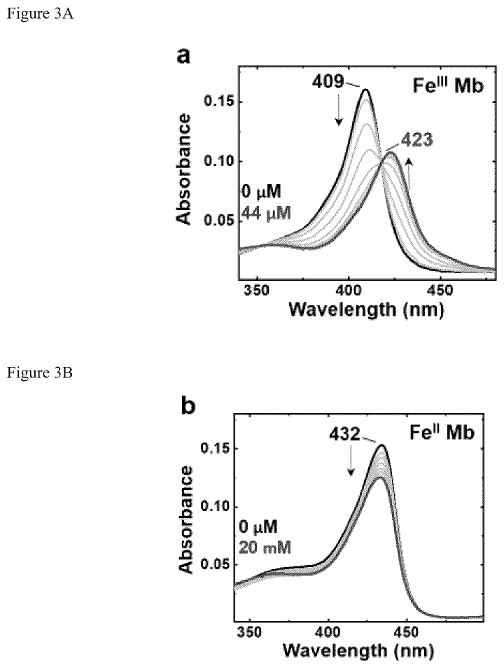

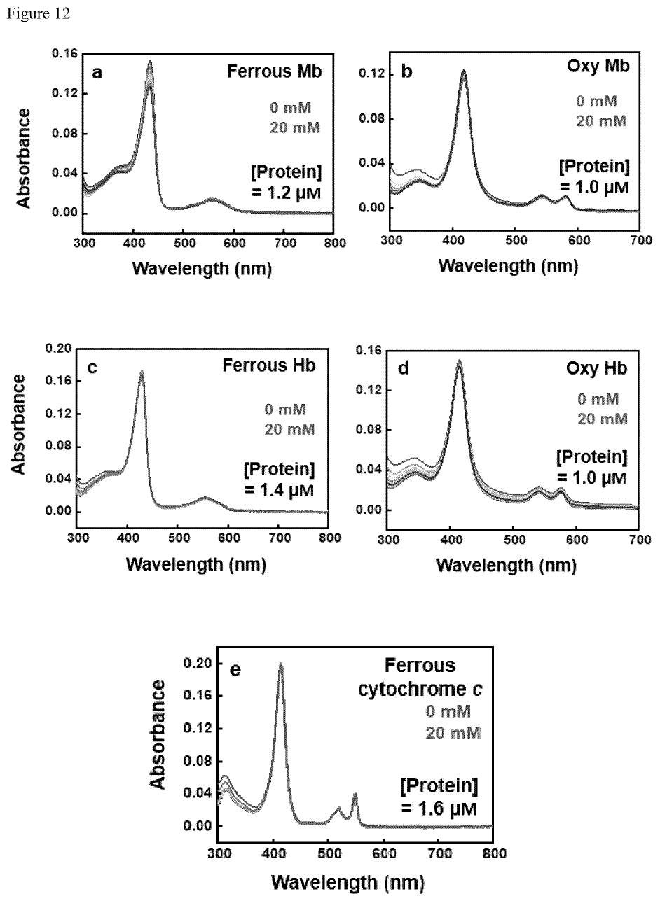

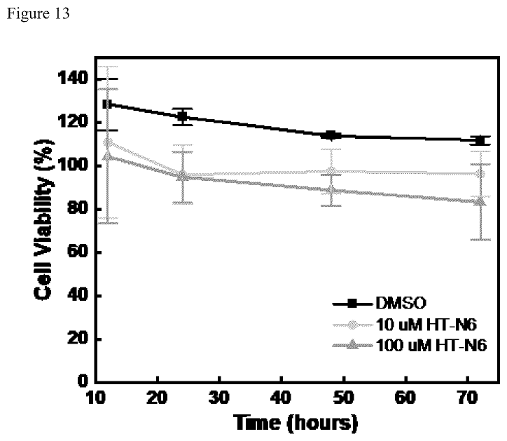

In studies thus far, the nature of branching moieties of nitrile tethers in tandem with the interior topology of heme protein impact tethering has been studied. A pertinent aspect that needs elucidation is the molecular basis of how the nitrile group (RC≡ [symbol] ) acts as a ligand to heme iron. It is possible that the nitrile N atom can act as a Lewis base by donating its sp-hybridized lone pair electrons to the Fe³⁺ of heme which is an electron deficient Lewis acid. Such a Lewis acid-base interaction will be significantly diminished for Fe²⁺ heme. A comparison of HT-N6 affinity studies on ferric versus ferrous Mb (FIGS. 3A and 3B, respectively) confirms such an expectation, and no binding to ferrous Mb is observed even at HT-N6 concentrations up to 20 mM. Such extreme selectivity of nitrile tethers between ferric and ferrous forms of heme is advantageous and can overcome the major hurdle arising from the promiscuity of myoglobin and haemoglobin towards the design of metal-binding here protein inhibitors. To illustrate such potential, the results of HT-N6 binding affinity measurements to tour different heme proteins in their physiologically relevant form (Table 1, FIG. 12) have been summarized. The HT-N6 fragment binds to the physiologically relevant ferric form of active M. tuberculosis's DosS protein, a high-value drug target, with a K_{d}=1.1±0.4 μM that is already comparable to the EC₅₀ (0.54 μM) (Zheng, H., et al., ACS Chem. Biol. 411793, 2019, doi:10.1101/411793; Zheng, H., et al., Nat. Chem. Biol. 13, 218-225, 2017) of the best-known inhibitor of DosS. At the same time, it exhibits no binding to the physiologically relevant forms of cytochrome c (ferric/ferrous), Hb (ferrous/ferrous-oxy), and Mb (ferrous/ferrous-oxy) that are ubiquitous in our body and essential for normal biological function.

TABLE 1
Effective K_{d} values of nitrile compound HT-N6 with different
heme proteins under physiological conditions

| | | Physiological | Physiological | Effective K_{d} | |
|---|---|---|---|---|---|
| Protein | Origin Species | redox state | Role | value | |
| Myoglobin | horse heart | Ferrous | O₂ carrier | >20 | mM |
| | | Ferrous-O₂ | | >20 | mM |
| Hemoglobin | human | Ferrous | O₂ carrier | >20 | mM |
| | | Ferrous-O₂ | | >20 | mM |
| Cytochrome c | equine heart | Ferrous | Electron | >20 | mM |
| | | | transport | | |
| DosS | M. Tuberculosis | Ferric | Gas signaling | 1.1 ± 0.4 | μM |
| | (active form) | | | | |

### Conclusion

Heme enzymes are involved in a variety of biological processes and have emerged as important targets for designing next-generation drugs (Poulos, T. L., Chem. Rev. 114, 3919-3962, 2014). However, these enzymes have remained undruggable so far since selective targeting of therapeutically relevant heme enzymes against numerous other ubiquitous heme proteins is challenging. Nitrile-based heme tethering offers a new modality to selectively target therapeutically relevant heme enzymes. The affinity of nitrile-based heme tethers can be modulated via various knobs including the nature, stereochemistry, and flexibility of branching moieties as well as incorporation of hydrogen-bonding interaction. At the same time, heme oxidation state, chemical and structural topologies of interior gas tunnels can be exploited to design highly specific heme enzyme tethers. Modulation of all of these knobs has resulted in a nitrile based heme tether (HT-N6) that binds to physiologically relevant ferric heme of active M. tuberculosis's DosS with >20,000-fold higher affinity over Hb, Mb, and cytochrome c which are predominant proteins in mammals (Table 1). Notably, DosS is an important drug target and inhibition of this sensor-kinase has been linked with blocking of M. tuberculosis from transitioning into its dormant and drug-resistant state (Sivaramakrishnan, S., et al., Biosensors 3, 259-282, 2013; Madrona, Y., et al., Arch. Biochem. Biophys. 612, 1-8, 2016). Overall, heme tethering is a highly efficient approach to selectively target heme proteins of therapeutic relevance and opens up drug discovery for not just heme proteins but other metalloproteins as well. It is anticipated that structurally-guided and rationally-designed nitrile-based heme tethers would find wide applications towards blocking biological functions of varied therapeutic heme proteins that are physiologically prevalent in their ferric form such as DosS, peroxidases, and P450s.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method to inhibit a metalloprotein comprising contacting the metalloprotein with a compound of formula I:
X-L-Y I
or a salt thereof, wherein:
X is nitrile (—CN), hydrazine, —SCN, —NCS, or —OCN;
L is a linker or L is absent; and
Y is a moiety that binds to a gas tunnel or a substrate tunnel of the metalloprotein.

2. The method of claim 1, wherein X is nitrile or hydrazine.

3. The method of claim 1, where X is nitrile.

4. The method of claim 1, wherein L is (C₁-C₈)alkyl, (C₃-C₇)carbocyclyl, —(C₁-C₆)alkyl(C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroalyl, wherein any (C₁-C₈)alkyl, (C₃-C₇)carbocyclyl, —(C₁-C₆)alkyl(C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of L is optionally substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl, and
wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

5. The method of claim 4, wherein L is (C₃-C₇)carbocyclyl, heterocyclyl, —(C₁-C₆)alkylheterocyclyl, or aryl, wherein any (C₃-C₇)carbocyclyl, heterocyclyl, —(C₁-C₆)alkylheterocyclyl, or aryl of L is optionally substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and
wherein each R^{L} is independently (C₁-C₁₂)alkyl, (C₁-C₁₂)heteroalkyl, or (C₃-C₇)carbocyclyl wherein the (C₇-C₁₂)alkyl, (C₁-C₁₂)heteroalkyl, or (C₃-C₇)carbocyclyl is optionally substituted with one or more halo.

6. The method of claim 4, wherein L is heterocyclyl or —(C₁-C₆)alkylheterocyclyl, wherein any heterocyclyl or —(C₁-C₆)alkylheterocyclyl of L is substituted with one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and
wherein each R^{L} is independently (C₁-C₁₂)alkyl, (C₁-C₁₂)heteroalkyl, or (C₃-C₇)carbocyclyl wherein the (C₁-C₁₂)alkyl, (C₃-C₁₂)heteroalkyl, or (C₃-C₇)carbocyclyl is optionally substituted with one or more halo.

7. The method of claim 4, wherein L is: 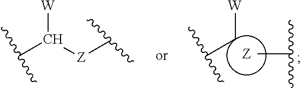
wherein;
W is a absent, H, —OH, —SH, —NH₂, CO₂H, or a 4-7 membered heterocycle; and
Z is a (C₃-C₇)carbocyclyl, aryl, or heterocycle, wherein the (C₃-C₇)carbocyclyl, aryl, or heterocycle is optionally substituted with one or more groups substituents independently selected from the one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and
wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

8. The method of claim 4, wherein L is: 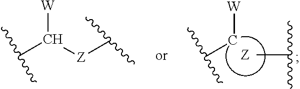
wherein;
W is a H, —OH, —SH, —NH₂, CO₂H, or a 4-7 membered heterocycle; and
Z is a heterocycle, wherein the heterocycle is optionally substituted with one or more groups substituents independently selected from the one or more substituents independently selected from the group consisting of halo, —OH, —OR^{L}, —SH, —SR^{L}, NH₂, NHR^{L}, —NR^{L}₂, CO₂H, CO₂R^{L}, (C₁-C₆)alkyl, (C₂-C₆)alkenyl, (C₂-C₆)alkynyl, 4-7 membered heterocyclyl, and (C₃-C₇)carbocyclyl, wherein (C₁-C₆)alkyl, (C₂-C₆)alkenyl, (C₂-C₆)alkynyl, 4-7 membered heterocyclyl, and (C₃-C₇)carbocyclyl is optionally substituted with one or more halo or (C₁-C₄)alkyl; and
wherein each R^{L} is independently (C₁-C₁₂)alkyl, (C₁-C₁₂)heteroalkyl, or (C₃-C₇)carbocyclyl wherein the (C₁-C₁₂)alkyl, (C₁-C₁₂)heteroalkyl, or (C₃-C₇)carbocyclyl is optionally substituted with one or more halo.

9. The method of claim 4, wherein L is: 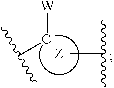
wherein;
W is a —OH, —SH, —NH₂, CO₂H, or a 4-7 membered heterocycle; and
Z is a heterocycle.

10. The method of claim 4, wherein L is: 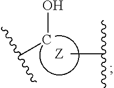
wherein Z is a heterocycle.

11. The method of claim 1, wherein L is absent.

12. The method of claim 1, wherein Y is (C₁-C₈)alkyl, (C₃-C₇)carbocyclyl, heterocyclyl, —(C₁-C₆)alkylheterocyclyl, aryl, —(C₁-C₆)alkylaryl, heteroaryl, —(C₁-C₆)alkylheteroaryl, CO₂H, CO₂R^{Z}, or —OR^{Z}, wherein any (C₁-C₈)alkyl, (C₃-C₇)carbocyclyl, heterocyclyl, —(C₁-C₆)alkylheterocyclyl, aryl, —(C₃-C₆)alkylaryl, heteroaryl, or —(C₁-C₆)alkylheteroaryl of Y is optionally substituted with one or more substituents independently selected from the group consisting of halo, —OH, —OR^{Y}, —SH, —SR^{Y}, NH₂, NHR^{Y}, —NR^{Y}₂, CO₂H, CO₂R^{Y}, (C₁-C₆)alkyl, (C₂-C₆)alkenyl, (C₂-C₆)alkynyl, 4-7 membered heterocyclyl, (C₃-C₇)carbocyclyl, and aryl, wherein (C₁-C₆)alkyl, (C₂-C₆)alkenyl, (C₂-C₆)alkynyl, 4-7 membered heterocyclyl, (C₃-C₇)carbocyclyl and aryl is optionally substituted with one or more halo or (C₁-C₄)alkyl;
wherein each R^{Y} is independently (C₁-C₁₂)alkyl, (C₁-C₁₂)heteroalkyl, or C₃-C₇carbocyclyl wherein the (C₁-C₁₂)alkyl, (C₁-C₁₂)heteroalkyl, or (C₃-C₇)carbocyclyl is optionally substituted with one or more halo; and
wherein each R^{Z} is independently (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl, wherein any (C₁-C₈)alkyl, (C₃-C₇)carbocyclyl, heterocyclyl, —(C₁-C₆)alkylheterocyclyl, aryl, —(C₁-C₆)alkylaryl, heteroaryl, or —(C₁-C₆)alkylheteroaryl of L is optionally substituted with one or more substituents independently selected from the group consisting of halo, —OH, —OR^{Y}, —SH, —SR^{Y}, NH₂, NHR^{Y}, —NR^{Y}₂, CO₂H, CO₂R^{Y}, (C₁-C₆)alkyl, (C₂-C₆)alkenyl, (C₂-C₆)alkynyl, 4-7 membered heterocyclyl, (C₃-C₇)carbocyclyl, and aryl.

13. The method of claim 1, wherein Y is —(C₁-C₆)alkylaryl, wherein —(C₁-C₆)alkylaryl, is optionally substituted with one or more substituents independently selected from the group consisting of halo, —OH, —OR^{Y}, —SH, —SR^{Y}, NH₂, NHR^{Y}, —NR^{Y}₂, CO₂H, CO₂R^{Y}, (C₁-C₆)alkyl, (C₂-C₆)alkenyl, (C₂-C₆)alkynyl, 4-7 membered heterocyclyl, (C₃-C₇)carbocyclyl, and aryl, wherein (C₁-C₆)alkyl, (C₂-C₆)alkenyl, (C₂-C₆)alkynyl, 4-7 membered heterocyclyl, (C₃-C₇)carbocyclyl and aryl is optionally substituted with one or more halo or (C₁-C₄)alkyl; and
wherein each R^{Y} is independently (C₁-C₁₂)alkyl, (C₁-C₁₂)heteroalkyl, (C₃-C₇)carbocyclyl wherein the (C₁-C₁₂)alkyl, (C₁-C₁₂)heteroalkyl, or (C₃-C₇)carbocyclyl is optionally substituted with one or more halo.

14. The method of claim 1, wherein the compound of formula I is a compound that is: 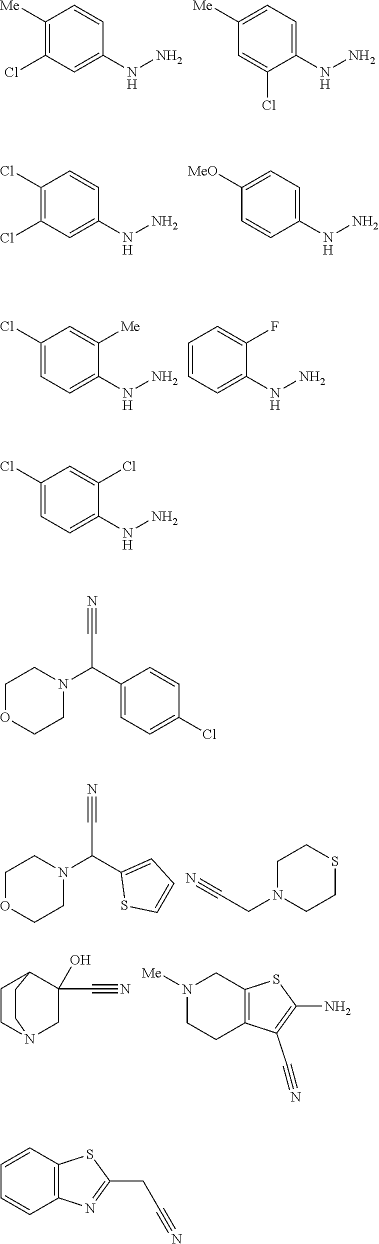 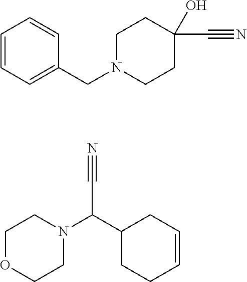 [SMILES: N#CC1(O)CCN(Cc2ccccc2)CC1 ; N#CC(C1CC=CCC1)N1CCOCC1]
or a salt thereof.

15. The method of claim 1, wherein the metalloprotein is a metalloenzyme.

16. The method of claim 1, wherein the metalloprotein is a heme enzyme.

17. The method of claim 1, wherein the metalloprotein is M. tuberculosis's DosS.

18. A method to treat a bacterial disease, viral disease, cardiovascular disease, neurological disease, or cancer in a mammal in need thereof, comprising administering a therapeutically effective amount of a compound as described in claim 1 or a pharmaceutically acceptable salt thereof to the mammal in need thereof.

19. The method of claim 18, wherein the disease is tuberculosis.

20. A method to treat a crop, to prevent infection of the crop, or to treat infection of the crop, comprising contacting the crop with a compound as described in claim 1 or a salt thereof, wherein the infection is a bacteria infection, an insect infection, a pest infection or another pathogen infection.
